(19) United States
(12) Patent Application Publication
(10) Pub. No.: US 2022/0225862 A1
(43) Pub. Date: Jul. 21, 2022

(54) ENDOSCOPE AND OVERTUBE

(71) Applicant: HOYA CORPORATION, Tokyo (JP)

(72) Inventor: Kunihiko ONOBORI, Tokyo (JP)

(73) Assignee: HOYA CORPORATION, Tokyo (JP)

(21) Appl. No.: 17/604,219

(22) PCT Filed: Sep. 4, 2020
(86) PCT No.: PCT/JP2020/033578

§ 371 Date: Oct. 15, 2021

(30) Foreign Application Priority Data
Sep. 30, 2019 (JP) 2019-178345

(51) Int. Cl. A61B 1/00; A61B 1/005; A61B 1/015; A61B 1/07; A61B 1/06

(52) U.S. Cl. CPC A61B 1/00135; A61B 1/0051; A61B 1/015; A61B 1/00087; A61B 1/0661; A61B 1/00117; A61B 1/07

(57) ABSTRACT

Provided is an endoscope system that inexpensively guides an extra-fine endoscope using an overtube. An endoscope system includes an endoscope and an overtube. The overtube includes an endoscope channel that allows passage of the endoscope, being bendable according to an operation. The endoscope includes a light irradiation unit and an imaging unit.

### TECHNICAL FIELD

The present invention relates to an endoscope and an overtube.

### BACKGROUND ART

An endoscope is used to observe the state of the digestive tract and the like inside the body. Observation of an extra-fine tract such as a lung requires an extra-fine endoscope that can be inserted into the extra-fine tract. In order to guide such an extra-fine endoscope to a part to be observed inside the body, an endoscope with a larger diameter (for example, an ordinary endoscope) may be used as a base endoscope together with the extra-fine endoscope.

The base endoscope is provided with a channel that allows passage of the extra-fine endoscope. The extra-fine endoscope having the function of an image sensor of the base endoscope is disposed inside the channel and inserted into a body lumen together with the base endoscope. After reaching an extra-fine tract such as a lung, the extra-fine endoscope is moved forward inside the channel and is protruded out of the base endoscope. Then, the extra-fine endoscope is further inserted into the periphery of the extra-fine tract. Extra-fine tracts are observed in this manner.

Patent Literature 1 discloses an example of an extra-fine endoscope system including these two endoscopes.

### CITATION LIST

### Patent Literature

Patent Literature 1: JP 2009-530051 A

### SUMMARY OF INVENTION

### Technical Problem

However, a base endoscope in an extra-fine endoscope system in the related art is designed to be reused, and using the base endoscope as a single-use (disposable) device is costly.

More specifically, despite the fact that the base endoscope is used only for guiding the extra-fine endoscope to a lesion area, one that has the function of an ordinary endoscope is used. Therefore, part of the function is wasted and the base endoscope becomes relatively expensive.

In addition, since configurations of the base endoscope and the extra-fine endoscope partially overlap each other (for example, image sensor, image processor, and light source), there is also a problem that the entire system increases in size.

Furthermore, reuse of the base endoscope as in the related art requires cleaning and sterilization after each use and requires labor and cost.

The present invention has been made to solve such problems. An object of the present invention is to provide an endoscope capable of guiding an extra-fine endoscope at low cost using an overtube.

### Solution to Problem

An endoscope system according to the present invention includes: an endoscope; and an overtube,

in which the overtube includes an endoscope channel that allows passage of the endoscope, the overtube being bendable according to an operation, and

the endoscope includes a light irradiation unit and an imaging unit.

This specification is based on and claims priority pursuant to Japanese Patent Application No. 2019-178345, the entire disclosure of which is hereby incorporated by reference herein.

### Advantageous Effects of Invention

An endoscope and an overtube according to an embodiment of the present invention makes it possible to inexpensively guide an extra-fine endoscope using the overtube.

### BRIEF DESCRIPTION OF DRAWINGS

FIG. 1 is a block diagram illustrating a partial configuration of an endoscope system according to a first embodiment.

FIG. 2 specifically illustrates a configuration of an insertion portion in FIG. 1.

FIG. 3 more specifically illustrates the configuration of the insertion portion in FIG. 1.

FIG. 4 illustrates a specific example of a configuration of an operation unit in FIG. 1.

FIG. 5 is a cross-sectional view of a connection taken along line V-V in FIG. 4.

FIG. 6 illustrates another example of a connection structure between an overtube operation unit and an endoscope operation unit in FIG. 4.

FIG. 7 is an enlarged view of a fixation between the overtube operation unit and the endoscope operation unit in FIG. 6.

FIG. 8 illustrates a state where the overtube operation unit and the endoscope operation unit in FIG. 6 are fixed to each other.

FIG. 9 illustrates still another example of the connection structure between the overtube operation unit and the endoscope operation unit in FIG. 4.

FIG. 10 illustrates a modification of a light irradiation unit in an overtube.

FIG. 11 illustrates a modification where the light irradiation unit of the overtube is omitted.

FIG. 12 illustrates another modification of the light irradiation unit in the overtube.

### DESCRIPTION OF EMBODIMENTS

Hereinafter, embodiments of the present invention will be described with reference to the accompanying drawings.

### First Embodiment

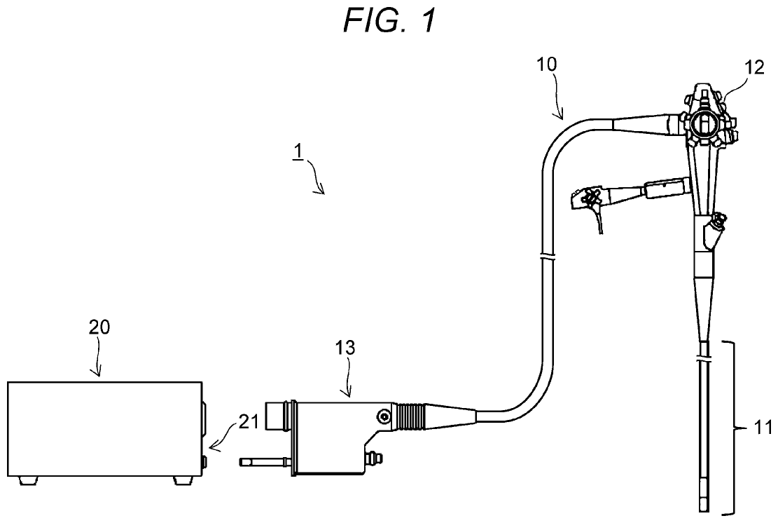

FIG. 1 is a block diagram illustrating a configuration of an endoscope system 1 used in this embodiment. The endoscope system 1 may be what is called an electronic endoscope. As illustrated in FIG. 1, the endoscope system 1 is, for example, a dedicated system for medical use and includes an endoscope unit 10 and a processor 20 (endoscope processor).

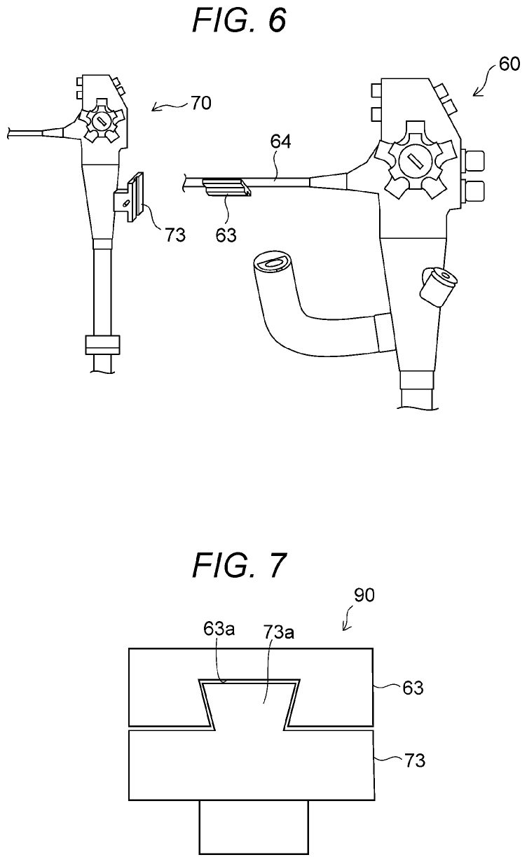

As illustrated in FIG. 1, the endoscope unit 10 includes an insertion portion 11 and an operation unit 12. Note that FIG. 1 conceptually illustrates the outline of functions of the operation unit 12 and does not necessarily accord with the actual configuration. A specific example of the configuration of the operation unit 12 will be described later with reference to FIGS. 6 to 9.

A user of the endoscope system 1 operates the operation unit 12 to control a movement of the insertion portion 11. For example, the insertion portion 11 is bent or curved according to an operation of the operation unit 12. Such a bending mechanism is a known mechanism incorporated in a typical electronic scope. For example, a knob included in the operation unit 12 is rotated, and in conjunction with the rotation, an operation wire is pulled, thereby bending a bendable portion of the insertion portion 11.

In the insertion portion 11, a part including the distal end can be inserted into any body lumen in a living organism. For example, the part is inserted into a bronchus, a biliary tract, a pancreas, a hepatic duct region, or a urinary organ region.

The processor 20 integrally includes a signal processing device that processes an image signal from the endoscope unit 10 and a light source device that applies light via the endoscope unit 10 to a body lumen which has no access to natural light. In another embodiment, the signal processing device and the light source device may be separated.

The proximal end of the endoscope unit 10 is provided with a connector unit 13, and the processor 20 is provided with a connector unit 21. The connector unit 13 and the connector unit 21 each have a coupling structure corresponding to each other. Coupling of these structures electrically and optically connects the endoscope unit 10 and the processor 20.

The processor 20 functions as a controller that controls the entire endoscope system 1 and includes, for example, a computer provided with an arithmetic unit and a storage unit. Other functions and configurations of the endoscope unit 10 and the processor 20 (for example, the function of acquiring an image inside a body lumen or the like) are appropriately designed by those skilled in the art based on known techniques. For example, the processor 20 performs various calculations based on specific information of the endoscope unit 10 and generates a control signal. Furthermore, the processor 20 uses the generated control signal to control operations and operation timings of various circuits inside the processor 20, thereby causing the endoscope unit 10 to move appropriately.

Note that the endoscope system 1 does not necessarily include the processor 20. Assuming that the endoscope unit 10 is connected to another appropriate processor, the endoscope unit 10 may be provided as an independent device.

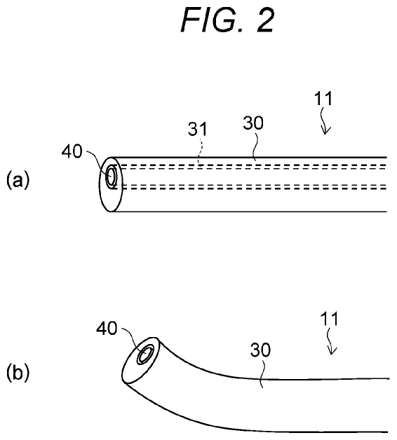

FIG. 2 specifically illustrates a configuration of the insertion portion 11. FIG. 2(a) illustrates a structure of the insertion portion 11, and FIG. 2(b) illustrates a state where the insertion portion 11 is bent. The endoscope system 1 includes an overtube 30 and an endoscope 40. The overtube 30 covers the outer periphery of the endoscope 40. The overtube 30 includes an endoscope channel 31. The endoscope channel 31 is, for example, disposed inside the overtube 30 as a cylindrical tubular space extending in the longitudinal direction (axial direction) of the overtube 30. The endoscope channel 31 may be what is called an endoscope tube and can be used as a channel that allows passage of the endoscope 40.

The overtube 30 and the endoscope 40 may be designed to have any size (such as diameter). However, using an endoscope with a smaller diameter (such as an extra-fine endoscope) as the endoscope 40 reduces the diameter of the entire insertion portion 11. For example, the overtube 30 has a diameter of 3 mm to 5 mm, and the endoscope 40 has a diameter of 1 mm.

The overtube 30 is bendable as illustrated in FIG. 2(b) according to a user's operation. The user operates the overtube 30, for example, by the operation unit 12. Although a specific configuration for bending the overtube 30 will not be described, those skilled in the art, for example, can determine an appropriate configuration based on known techniques using an operation wire or the like. Note that the overtube 30 and the endoscope 40 may include a non-bendable rigid portion. In this case, a bendable portion other than the rigid portion is bent.

As illustrated in FIG. 2(b), since the endoscope 40 is disposed inside the endoscope channel 31, the endoscope 40 is bent when the overtube 30 bends. In other words, the overtube 30 is actively bendable while the endoscope 40 is passively bendable.

As a modification, for example, in a case where the endoscope 40 is allowed to increase in diameter, the endoscope 40 is bent or curved according to an operation of the operation unit 12. In this case, the overtube 30 may be passively bent or curved. The endoscope 40 may be bent in one direction (for example, in the horizontal direction or the vertical direction) or may be bent in a plurality of directions (for example, the vertical direction and the horizontal direction similarly to the overtube 30).

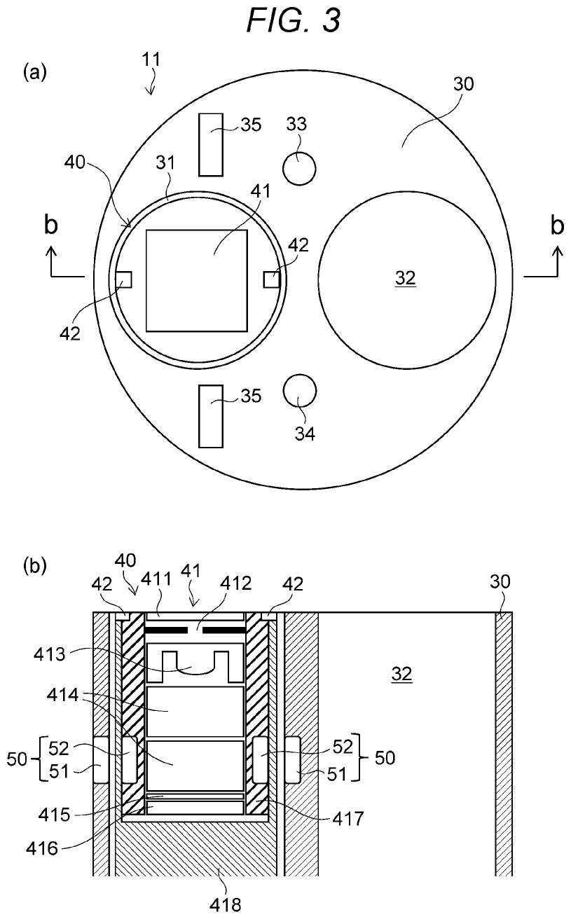

FIG. 3 more specifically illustrates the configuration of the insertion portion 11. FIG. 3(a) is an end view of the distal tip (distal end) of the insertion portion 11, and FIG. 3(b) is a partial cross-sectional view taken along line b-b of FIG. 3(a).

The overtube 30 may include a forceps channel 32. The forceps channel 32 may be what is called a forceps tube and can be used as a channel that allows passage of a forceps (including treatment tools and other required devices). Furthermore, the forceps channel 32 may be used to allow passage of an endoscope different from the endoscope 40. Still further, the forceps channel 32 can also be used for suction in order to remove minute obstacles or the like from the inside of a living organism. The forceps channel 32 is, for example, disposed inside the overtube 30 as a cylindrical tubular space extending in the longitudinal direction of the overtube 30.

The overtube 30 may include an air/water supply channel 33. The air/water supply channel 33 is used as a passage for carrying a fluid (for example, air or other gases, or water or other liquids). The air/water supply channel 33 is, for example, disposed inside the overtube 30 as a cylindrical tubular space extending in the longitudinal direction of the overtube 30. The fluid is carried toward the distal end through the air/water supply channel 33.

The fluid carried by the air/water supply channel 33 may be used to clean the distal surface of the endoscope 40. For this purpose, the distal tip of the air/water supply channel 33 may be provided with a nozzle, and the nozzle may be configured to eject a fluid toward the endoscope 40.

The overtube 30 may include a water jet channel 34. The water jet channel 34 is used as a passage for carrying a liquid (water or other liquids). The water jet channel 34 is, for example, disposed inside the overtube 30 as a cylindrical tubular space extending in the longitudinal direction of the overtube 30. The liquid is carried through this water jet channel 34 towards the distal end.

The liquid carried by the water jet channel 34 may be used to clean a living organism. For example, minute obstacles or the like inside a body lumen can be cleaned away and removed by the liquid.

The overtube 30 may include a light irradiation unit 35. In this embodiment, the light irradiation unit 35 includes a light emitting diode (LED). The light irradiation unit 35 provides illumination light necessary for capturing an image by the endoscope unit 10. The light irradiation unit 35 is placed at or near the distal tip of the overtube 30.

The endoscope 40 is disposed inside the endoscope channel 31 of the overtube 30. The endoscope 40 includes an imaging unit 41. The imaging unit 41 may be configured as an imaging unit of a known endoscope, but an example of the configuration will be described with reference to FIG. 3(b).

The imaging unit 41 includes a transparent cover 411, an aperture 412, an objective optical system including a lens 413 (for example, a convex lens) and one or more spacers 414, a cover glass 415, and an image sensor 416 (for example, a CMOS sensor). These components are arranged in a hollow cylindrical rod 417. These structures including the rod 417 are disposed inside a hollow cylindrical outer tube 418.

The endoscope 40 includes a light irradiation unit 42. The light irradiation unit 42 is placed at or near the distal tip of the endoscope 40. The light irradiation unit 42 is, for example, a light guide and includes an optical fiber in this embodiment. In an example illustrated in FIG. 3, the optical fiber is supported by a light guide holder fixed to the distal tip (distal end) of the endoscope 40. In the light irradiation unit 42 illustrated in FIG. 3, the optical fiber and the light guide holder are not distinguished from each other.

Although not illustrated in FIG. 3, the endoscope 40 may include wires as appropriate. For example, along the outer circumference of the rod 417, the optical fiber extends into the inside of the endoscope channel 31 or the endoscope 40 and provides light from the light source inside the processor 20 to the distal tip. Furthermore, for example, a sensor cable for transmitting a signal may be connected to the image sensor 416. The sensor cable may extend into the inside of the endoscope channel 31 or the endoscope 40 and transmit a signal that represents an image to the processor 20.

In this embodiment, the overtube 30 does not include an imaging unit. Accordingly, the overtube 30 can be produced relatively inexpensively and produced to have a configuration preferable as a single-use device.

The overtube 30 protects and guides the endoscope 40 until the endoscope unit 10 reaches an extra-fine tract inside the body, providing the function that the endoscope 40 does not have. In other words, the overtube 30 provides a function obtained by subtracting the function of the endoscope 40 according to this embodiment from the function of an endoscope in the related art. Therefore, the endoscope 40 does not necessarily have all the functions that an endoscope in the related art standardly has.

The endoscope system 1 includes an endoscope fixation 50 for releasably fixing the overtube 30 and the endoscope 40. In this embodiment, the endoscope 40 is disposed inside the overtube 30, and the overtube 30 prevents the movement of the endoscope 40 in the radial direction. Accordingly, the endoscope fixation 50 may be one that fixes the overtube 30 and the endoscope 40 in the longitudinal direction.

The endoscope fixation 50 is placed, for example, within a range of 3 mm to 15 mm from the distal tip of the endoscope unit 10. In a case where the non-bendable rigid portion is disposed close to the distal tip of the endoscope unit 10, the endoscope fixation 50 may be disposed in the rigid portion. However, the endoscope fixation 50 is not limited to the position.

In this embodiment, the endoscope fixation 50 includes a permanent magnet. The endoscope fixation 50 includes an N-pole magnet 51 fixed to the overtube 30 and an S-pole magnet 52 fixed to the endoscope 40. At least one N-pole magnet 51 and at least one S-pole magnet 52 are disposed to face each other, thereby fixing the overtube 30 and the endoscope 40 to each other by a magnetic attractive force.

Relative positions of the overtube 30 and the endoscope fixation 50 are fixed by the endoscope fixation 50. Particularly, the positional relation between the distal surface of the overtube 30 and the distal surface of the endoscope fixation 50 is fixed. When the endoscope fixation 50 is configured to align the longitudinal positions of the distal surfaces (for example, to make the distal surfaces coplanar), it is possible to provide the functions of the overtube 30 and the endoscope 40 in a complementary manner. For example, it is possible to operate a treatment tool via the overtube 30 or supply air or water while capturing and checking an image by the endoscope 40. Accordingly, the insertion portion 11 including the overtube 30 and the endoscope 40 can be operated similarly to an insertion portion of an endoscope in the related art, which makes it possible to provide functions similar to endoscopes in the related art.

When a force that exceeds the magnetic attractive force and separates the N-pole magnet 51 and the S-pole magnet 52 acts between the N-pole magnet 51 and the S-pole magnet 52, the correlative fixing between the overtube 30 and the endoscope 40 is unfixed. For example, when a force exceeding a predetermined threshold acts to push the endoscope 40 toward the distal tip, the endoscope 40 is disengaged from the overtube 30 and moves toward the distal tip. Such a force may be generated according to a specific operation of the operation unit 12.

In this embodiment, after disengagement of the overtube 30 and the endoscope 40, it is possible to fix the overtube 30 and the endoscope 40 again. In other words, pulling back the endoscope 40 to the fixed position causes a magnetic attractive force of the N-pole magnet 51 and the S-pole magnet 52, thereby fixing the overtube 30 and the endoscope 40 again.

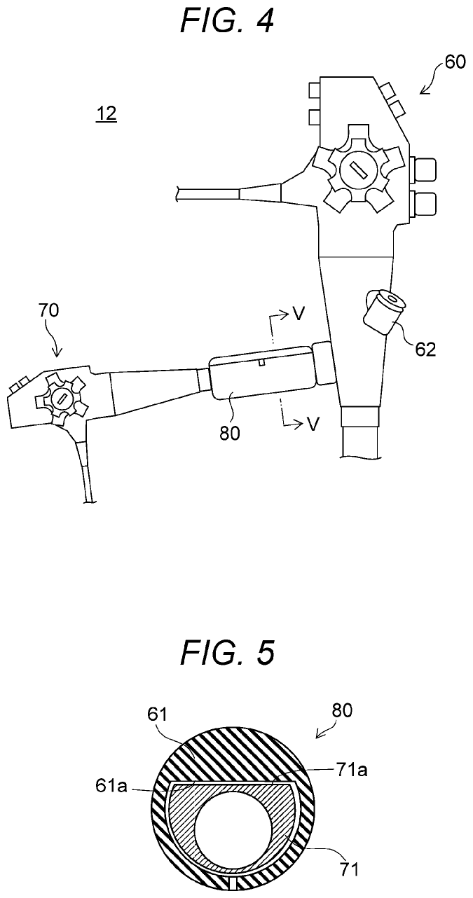

FIG. 4 illustrates a specific example of the configuration of the operation unit 12. The operation unit 12 includes an overtube operation unit 60 and an endoscope operation unit 70. The overtube operation unit 60 is for operating the overtube 30, and the endoscope operation unit 70 is for operating the endoscope 40. The overtube operation unit 60 and the endoscope operation unit 70 are connected to each other in a connection 80. The connection 80 may be a part of the overtube operation unit 60, a part of the endoscope operation unit 70, or a combination of both parts.

The overtube 30 and the endoscope 40 are connected in the overtube operation unit 60, the endoscope operation unit 70, or the connection 80. For example, inside the overtube operation unit 60, the endoscope operation unit 70, or the connection 80, the endoscope channel 31 of the overtube 30 opens toward the outer periphery of the overtube 30 to form an insertion port for allowing insertion of the endoscope 40. The endoscope 40 is integrated with the overtube 30 by being inserted into the insertion port. Accordingly, the endoscope 40 can be inserted into the body.

The overtube operation unit 60 includes an operational tool for operating the overtube 30. Although a specific structure of the operational tool is not particularly described in relation to each part illustrated in FIG. 4, for example, the overtube operation unit 60 includes a knob for bending the overtube 30 vertically and a knob for bending the overtube 30 horizontally. In addition, the overtube operation unit 60 may include, for example, an air/water supply button for supplying a fluid through the air/water supply channel 33, a water jet button for supplying a liquid through the water jet channel 34, and a suction button for suctioning through the forceps channel 32.

Although not particularly illustrated, the operation unit 12 may include an operation panel for inputting information or instructions. The operation panel may include a hardware key, a touch panel GUI, or a combination of a hardware key and a touch panel GUI.

The overtube operation unit 60 also includes a forceps channel inlet 62. The forceps channel inlet 62 is included in the proximal end of the forceps channel 32 and is configured to allow insertion of treatment tools, necessary devices, and the like.

The endoscope operation unit 70 includes an operational tool for operating the endoscope 40. Although a specific structure of the operational tool is not particularly described in relation to each part illustrated in FIG. 4, for example, the endoscope operation unit 70 includes an imaging button for controlling imaging by the imaging unit 41 and a light control button for controlling light irradiation by the light irradiation unit 42. Furthermore, the endoscope operation unit 70 may include a disengagement button for unfixing the fixing by the endoscope fixation 50. Still further, in a case where the endoscope 40 has the bending function, the endoscope operation unit 70 may include, for example, a knob for bending the endoscope 40 vertically and a knob for bending the endoscope 40 horizontally.

Note that FIG. 4 mainly illustrates the function of the operation unit 12 and does not necessarily accurately represent the actual shape. For example, connection angles of the forceps channel inlet 62 and the endoscope operation unit 70 relative to the overtube operation unit 60 can be appropriately designed depending on the strength, flexibility, and the like of the overtube 30 and the endoscope 40.

FIG. 5 is a cross-sectional view of the connection 80 taken along line V-V in FIG. 4. In this example, the connection 80 is configured by combining a connecting member 61 of the overtube operation unit 60 and a connecting member 71 of the endoscope operation unit 70. The overtube operation unit 60 and the endoscope operation unit 70 are connected by inserting and fitting the connecting member 71 into the connecting member 61.

In this example, a flat portion 61a is formed on the inner periphery of the outer connecting member 61, and a flat portion 71a is formed on the outer periphery of the inner connecting member 71. As described above, in the connecting members 61 and 71, at least a part of each structure is not cylindrical. Accordingly, while the connecting member 71 is fitted into the connecting member 61, it is possible to prevent relative rotation.

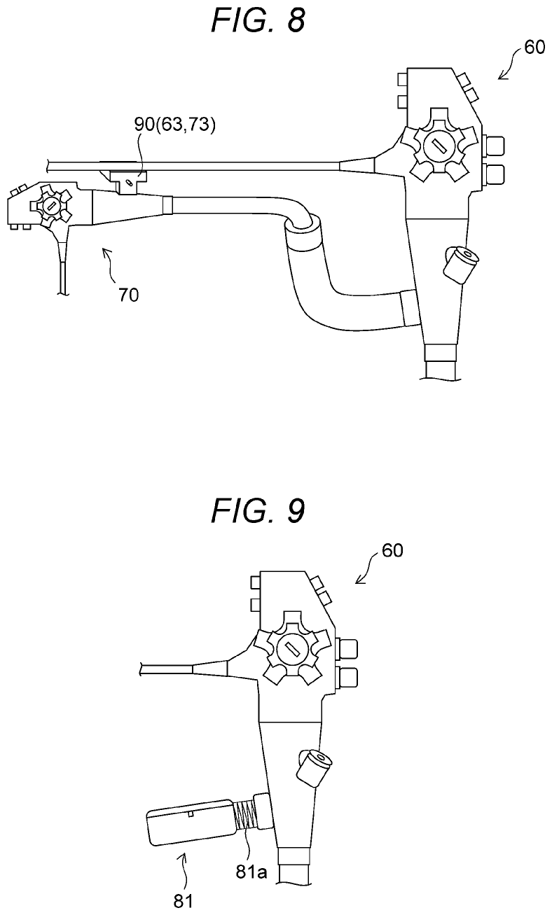

Another example of the connection structure between the overtube operation unit 60 and the endoscope operation unit 70 will be described with reference to FIGS. 6 to 8. FIG. 6 illustrates a state where the overtube operation unit 60 and the endoscope operation unit 70 are not fixed to each other. FIG. 7 is an enlarged view of an operation unit fixation 90. FIG. 8 illustrates a state where the overtube operation unit 60 and the endoscope operation unit 70 are fixed to each other.

As illustrated in FIG. 6, the overtube operation unit 60 includes a fixing member 63, and the endoscope operation unit 70 includes a fixing member 73. The fixing member 63 is fixed to, for example, a cover tube 64 that covers the overtube 30. The fixing member 73 is fixed to, for example, the endoscope operation unit 70.

In this manner, when the fixing members 63 and 73 are not engaged with each other, the overtube operation unit 60 and the endoscope operation unit 70 are not fixed to each other. Within a range allowed by the overtube 30 and the endoscope 40, it is possible to change relative positions and postures freely.

The fixing members 63 and 73 can be engaged as illustrated in FIG. 7. A recess 63a of the fixing member 63 and a protrusion 73a of the fixing member 73 are slid in the depth direction of FIG. 7 (from this side of the paper to the other side) and engaged with each other. Accordingly, the fixing members 63 and 73 in FIG. 7 are prevented from moving in a relative manner and are fixed. FIG. 8 illustrates the overtube operation unit 60 and the endoscope operation unit 70 fixed in this manner. In order to unfix the fixing members 63 and 73, the fixing members 63 and 73 may be slid and disengaged in the depth direction of FIG. 7.

According to the configuration as illustrated in FIGS. 6 to 8, since the endoscope operation unit 70 can be releasably fixed to the overtube operation unit 60, the endoscope operation unit 70 can be appropriately held when the operation by the endoscope operation unit 70 is not necessary. For example, while the endoscope operation unit 70 is fixed to the overtube operation unit 60, it is possible to operate the overtube operation unit 60 to insert the overtube 30 into a lesion area, and then, detach the endoscope operation unit 70 from the overtube operation unit 60, and operate the endoscope operation unit 70 to control the endoscope 40.

Still another example of the connection structure between the overtube operation unit 60 and the endoscope operation unit 70 will be described with reference to FIG. 9 (note that the endoscope operation unit 70 is not illustrated in FIG. 9). The overtube operation unit 60 includes a connection 81 for connecting the endoscope operation unit 70. The connection 81 includes an adjuster 81a. A length of the adjuster 81a is adjustable and is achieved by, for example, a screw structure. The adjuster 81a can be used to adjust an insertion length of the endoscope 40 with respect to the endoscope channel 31 of the overtube 30.

Using such an adjuster 81a enables adjustment of the insertion length even when the endoscope unit 10 is inserted into the body. Accordingly, the distal tip of the overtube 30 and the distal tip of the endoscope 40 can be easily aligned at any time point. More specifically, for example, in a case where the distal tip of the overtube 30 and the distal tip of the endoscope unit 10 are misaligned after inserting or bending the endoscope 40, it is possible to align these distal tips again by operating the adjuster 81a at that time. With the adjuster 81a, note that the endoscope fixation 50 may be omitted.

In an example illustrated in FIG. 9, the adjuster 81a is disposed in the overtube operation unit 60, but the adjuster 81a is not limited to the position, and the adjuster 81a can be placed at any position along the endoscope 40. In the example of FIG. 9, an insertion length of the endoscope 40 with respect to the endoscope channel 31 changes along with the change in length of the adjuster 81a. However, the adjuster 81a does not necessarily change in length.

As described above, the overtube 30 and the endoscope system 1 according to the first embodiment of the present invention enables configuration of the overtube 30 at a relatively low cost, thereby providing the overtube 30 as a single-use device.

In addition, providing the overtube 30 as a single-use device removes the need for cleaning and sterilization after each use, which saves labor and cost. Particularly, sterilization before shipping the overtube 30 removes the need for sterilization at the site of use.

In addition, since the overtube 30 includes the endoscope channel 31 and the forceps channel 32 separately, the endoscope 40 and another treatment tool or the like can be used simultaneously.

The first embodiment can be modified in the following manner.

The overtube 30 can be provided as an independent member without being combined with the endoscope 40. In this case, the overtube 30 can be used together with an endoscope in the related art.

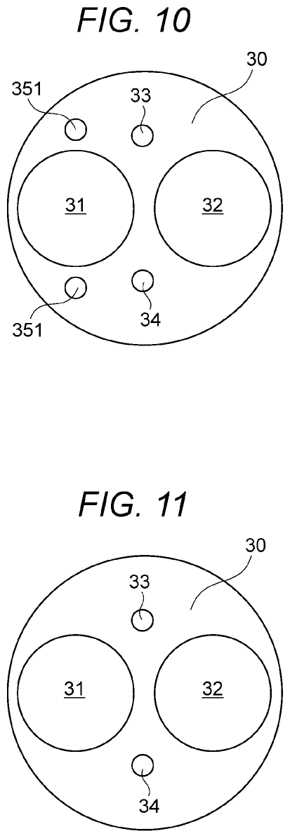

The light irradiation unit in the overtube 30 is not limited to an LED as the light irradiation unit 35 in FIG. 3. In a modification illustrated in FIG. 10, a light guide 351 is provided as the light irradiation unit. The light guide 351 is, for example, an optical fiber and emits light supplied from a separately provided light source (for example, the light source disposed inside the processor 20) at the distal tip of the overtube 30.

In a modification illustrated in FIG. 11, the light irradiation unit of the overtube 30 is omitted. Even in this case, it is possible to capture an image using illumination light since the endoscope 40 includes the light irradiation unit 42.

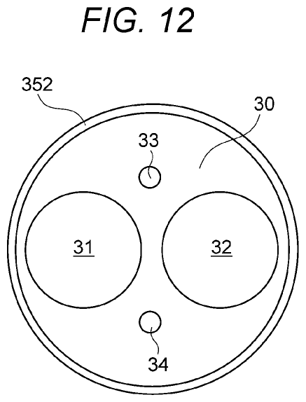

In a modification illustrated in FIG. 12, an annular light source 352 as the light irradiation unit of the overtube 30 is disposed along the outer periphery of the distal tip of the overtube 30. An example of the light source 352 includes a surface emitting type light source, and a more specific example is an organic light emitting diode (OLED).

Other components of the overtube 30 can also be omitted depending on the intended use. For example, one or all of the forceps channel 32, the air/water supply channel 33, and the water jet channel 34 may be omitted. The overtube 30 does not necessarily have all the configurations or functions that an endoscope in the related art standardly has.

The light irradiation unit in the endoscope 40 is not limited to a light guide as the light irradiation unit 42 in FIG. 3. An LED or OLED may be used.

Furthermore, the endoscope 40 may include a channel having the function similar to that of the forceps channel 32, the air/water supply channel 33, or the water jet channel 34.

The endoscope fixation 50 does not necessarily include a permanent magnet as illustrated in FIG. 3. It is possible to employ any known configuration capable of releasable fixing. For example, an electromagnet may be used. In this case, the overtube 30 and the endoscope 40 are fixed by applying a current to the electromagnet, and the overtube 30 and the endoscope 40 are unfixed by stopping the current. The current to the electromagnet may be controlled by the operation unit 12 or the processor 20.

Alternatively, an engagement structure using a recess and a protrusion may be employed. In other words, a protrusion may be formed on one of the inner periphery of the overtube 30 and the outer periphery of the endoscope 40, and a recess may be formed on the other, and these members may be engaged with each other to fix the overtube 30 and the endoscope 40. In a case where the structure is designed such that the protrusion and the recess are disengaged when a force exceeding a predetermined threshold is applied in the longitudinal direction, it is possible to unfix the protrusion and the recess optionally.

In the first embodiment, the endoscope fixation 50 can be fixed again after disengagement. As a modification, a fixation may be designed not to be fixed again.

Note that the endoscope system 1 or the endoscope 40 is not limited to the configurations and functions described in this specification and may have the configurations and functions of known endoscopes.

The present disclosure includes the following specific elements.

[Specific Element 1]

An endoscope system including: an endoscope; and an overtube,

in which the overtube includes an endoscope channel that allows passage of the endoscope, the overtube being bendable according to an operation, and

the endoscope includes a light irradiation unit and an imaging unit.

[Specific Element 2]

The endoscope system of specific element 1, in which the overtube includes a forceps channel.

[Specific Element 3]

The endoscope system according to specific element 1 or 2, the endoscope system including an endoscope fixation configured to fix the overtube and the endoscope releasably.

[Specific Element 4]

The endoscope system according to any one of specific elements 1 to 3, in which the overtube includes a passage for carrying a fluid.

[Specific Element 5]

The endoscope system according to any one of specific elements 1 to 4,

the endoscope system including an endoscope operation unit for operating the endoscope, and

the endoscope system including an operation unit fixation for releasably fixing the overtube and the endoscope operation unit.

[Specific Element 6]

The endoscope system according to any one of specific elements 1 to 5, the endoscope system including an adjuster configured to adjust an insertion length of the endoscope with respect to the endoscope channel.

[Specific Element 7]

An overtube including an endoscope channel that allows passage of an endoscope, the overtube being bendable according to an operation.

### REFERENCE SIGNS LIST

1 Endoscope system
10 Endoscope unit
11 Insertion portion
12 Operation unit
30 Overtube
31 Endoscope channel
32 Forceps channel
33 Air/water supply channel (passage)
34 Water jet channel (passage)
35 Light irradiation unit
40 Endoscope
41 Imaging unit
42 Light irradiation unit
50 Endoscope fixation
60 Overtube operation unit
70 Endoscope operation unit
81 Connection
81a Adjuster
90 Operation unit fixation

All publications, patents, and patent applications cited herein are hereby incorporated by reference in their entirety.

1. An endoscope system comprising: an endoscope; and an overtube,
wherein the overtube includes an endoscope channel that allows passage of the endoscope, the overtube being bendable according to an operation, and
the endoscope includes a light irradiation unit and an imaging unit.

2. The endoscope system of claim 1, wherein the overtube includes a forceps channel.

3. The endoscope system according to claim 1, the endoscope system comprising an endoscope fixation configured to fix the overtube and the endoscope releasably.

4. The endoscope system according to claim 1, wherein the overtube includes a passage for carrying a fluid.

5. The endoscope system according to claim 1,
the endoscope system comprising an endoscope operation unit for operating the endoscope, and
the endoscope system comprising an operation unit fixation for releasably fixing the overtube and the endoscope operation unit.

6. The endoscope system according to claim 1, the endoscope system comprising an adjuster configured to adjust an insertion length of the endoscope with respect to the endoscope channel.

7. An overtube comprising an endoscope channel that allows passage of an endoscope, the overtube being bendable according to an operation.
